(19) United States
(12) Patent Application Publication
(10) Pub. No.: US 2007/0260229 A1
(43) Pub. Date: Nov. 8, 2007

(54) Method and kit for treatment of varicose veins and other superficial venous pathology

(76) Inventors: Luis Navarro, New York, NY (US); Nestor Navarro, Barcelona (ES); Carlos Bone Salat, Mallorca (ES)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 11/524,553

(22) Filed: Sep. 20, 2006

Related U.S. Application Data
(60) Provisional application No. 60/798,359, filed on May 5, 2006.

(51) Int. Cl. A61B 18/18

(52) U.S. Cl. 606/9

(57) ABSTRACT

A method is disclosed for treating superficial venous pathology in a patient. The method comprises the steps of: (a) percutaneously piercing a vein to be treated; and (b) directing intense pulse or laser light at the patient's skin predominantly within the area of skin manifesting physical, chemical and/or color changes caused by step (a). In a preferred method sclerotherapy is performed on the vein to be treated and then laser light is directed at the patient's skin substantially entirely within the area of skin manifesting the changes.

### CROSS-REFERENCE TO RELATED APPLICATIONS

This application claims the benefit of U.S. Provisional Patent Application Ser. No. 60/798,359 filed May 5, 2006 which is hereby incorporated by reference in its entirety.

### FIELD OF THE INVENTION

The present invention relates to methods and kits for treating varicose and spider veins. The method combines a first step in which the patient's vein is pierced percutaneously followed by a second step in which light is directed at the area of the patient's skin affected by the piercing step. In a preferred method sclerotherapy is followed by a laser-light treatment. The method involving sclerotherapy collapses the unwanted veins more rapidly than either light treatment alone or sclerotherapy alone or known combinations of those treatments.

### DESCRIPTION OF PRIOR ART

Superficial venous branches, reticular veins, venectasia, telangiectases and other superficial venous pathology, may exist alone or as part of a more severe venous insufficiency with large truncal, valvular, and/or perforator involvement. Where venous insufficiency is present it is typically dealt with by surgical procedures, e.g. ligation and stripping, ligation alone or miniphlebectomy; by traditional or ultrasound guided sclerotherapy; or by endovenous laser or endovenous radiofrequency procedures before treating the superficial venous pathology.

Treatment of superficial venous pathology, in the absence of truncal venous pathology, or, after its treatment, has traditionally been done by the following techniques: sclerotherapy alone; percutaneous laser or light therapy alone; sclerotherapy in combination with percutaneous laser or light therapy; invasive paravenous or endovenous laser techniques; and electrocoagulation.

Sclerotherapy is considered the gold standard for the treatment of superficial venous pathology. Compared with light energy techniques, sclerotherapy is a simple technique, which covers a length of vein or area with a single injection and easily treats veins of different sizes, depth, color, pressure and tortuosity. Sclerotherapy has the advantage of being vein specific, and, when performed properly decreases hydrostatic venous pressure in a sequential manner in the affected veins. This is one of the most important points in its success and avoidance of unwanted side effects.

Sclerotherapy consists of the injection of a sclerosing agent into the vein to be treated. The sclerosing agent irritates the inner layers of the vein causing the vein to collapse. The treated area veins then heal by fibrosis. The procedure involves injecting a chemical sclerosing agent or combination of agents in various concentrations and volumes, into the lumen(s) of the targeted vein(s) at multiple locations to produce diffuse sclerosis. Good results are obtained when appropriate techniques and multiple sclerosing agents are used followed by post-sclerotherapy local compression.

Percutaneous laser or light therapy relies on the phenomenon of photocoagulation and is based on the principle of selective photothermolysis. The area containing the veins and/or the whole length of vein is irradiated with light, usually laser or intense pulse light (“IPL”). The wavelength of the light is chosen so that the light energy will be preferentially absorbed by the hemoglobin (Hb) in the targeted veins. The absorption leads to localized heating of the blood and increases the temperature of the veins to a point at which the constituent proteins denature and coagulate. Healing follows with closure of the vein. Multiple light wavelengths, pulse durations, pulse intervals, fluencies, and cooling devices are used in an effort to treat the variety of vein sizes, colors, depths and pressure.

Grove et al., U.S. Pat. No. 5,707,403, describe the use of laser energy to treat the whole area and/or length of the blood vessels. In Grove et al. laser light at a wavelength of 700-1100 mn is delivered at the surface of the skin. Blood vessels within the first 2 millimeters of the dermis are advantageously treated with light at this wavelength compared with light at shorter wavelengths which does not penetrate the dermis and therefore causes surface vessels to explode with attendant high absorption by melanin and burning of the skin. Although using light of 700-1100 nm offers advantages over light of shorter wavelengths, experience has shown the results in treating superficial venous pathology to be inferior to sclerotherapy.

In a more invasive variation of the percutaneous laser treatment of venous pathology, Trelles, U.S. Pat. No. 5,522,813, discloses the use of multiple pulses of a CO₂ laser to drill a small channel in the skin until the vessel is reached. The laser light coagulates and collapses the vein at that particular point. This procedure must be repeated multiple times to produce multiple interruptions of the vein.

Given the variability of size, depth, color and pressure etc. of superficial venous branches, reticular veins, venectasia, telangiectases and other superficial venous pathology, percutaneous, light or laser therapy treatment of these veins, even using light having a range of wavelengths, fluencies, pulse durations, intervals etc., has rarely achieved the results obtained with sclerotherapy. Skin complications e.g. hyper- and hypo-pigmentation, blisters, etc. have occurred due to the competition between the main chromophore in this methodology, Hb, and other skin chromophores such as melanin.

Combinations of sclerotherapy and percutaneous laser therapy have been used. Leg telangiectasias have been treated with laser light, before, immediately after, or after a delay—a dwell time—following the injection of a sclerosing agent. Goldman, et al, Sclerotherapy, 1995, at pages 454-458. The consensus of clinicians is that combination therapy brings the results of laser therapy to the level of success of sclerotherapy treatment, but, at the price of increased complications, complexity and cost without any added benefit to the use of sclerotherapy alone.

A recent study, Levy, Lasers in Surg & Med (2004), 34:237-276 reports that laser irradiation of a whole vein of 0.5-2 mm diameter with light of 1064 nm wavelength one month after sclerotherapy results in superior results compared to sclerotherapy or lasers alone. Cisnero, J. L., Dermatol. Surg.1998; 24;1119-1123, describes using laser therapy after a dwell time of 7 to 10 days following sclerotherapy. Furumoto et al., U.S. Pat. No. 5,843,072, use laser light treatment after a dwell time of 12 hours to 6 months following sclerotherapy and claim an increased success rate.

Invasive laser techniques (paravenous or endovenous) are methods in which a laser-emitting device is passed through the skin and placed next to the vein or into the lumen of the vein to degrade and collapse the vein. Trelles, U.S. Pat. No. 5,531,739, describes a procedure in which laser energy is delivered from below the skin. The Trelles patent teaches a method in which laser energy is delivered via a fiber optic probe to a location underneath a blood vessel to be treated. The vessel is irradiated with a laser beam having a fluence sufficient to coagulate and collapse the vessel at that location. This procedure must be repeated at multiple sites along the length of the blood vessel so that it will collapse along its length and no longer carry any blood.

Goldman, U.S. Pat. No. 4,564,011, delivers laser light beneath the skin via a hollow needle inserted within a blood vessel. The light energy creates a blood clot. Goldman also teaches using laser energy delivered subcutaneously with a laser fiber immediately adjacent a damaged blood vessel to create white scar tissue which tends to push against the vessel, thereby causing the vessel to shrink in size and at least partially disappear from view. This method requires that each single point of damage be treated separately.

Del Giglio, WO No. 0103596A1, describes a procedure in which laser energy is delivered from below the skin. The Del Giglio patent discloses a procedure in which laser energy is delivered endovenously through a handheld device to spider veins, feeder veins, and varicose veins, using laser fibers of different calibers. The target of the laser energy is the blood contained within the vessel. The laser energy creates a micro bubble explosion, which destroys the vein.

Navarro, et al, U.S. Pat. No. 6,398,777, describe endovenous delivery of laser energy via a bare tip laser fiber in contact with the wall of the vein to cause fibrosis of the treated blood vessel. The methodology is usually limited to main trunks and secondary branches, given laser fiber sizes and the difficulty in cannulating smaller veins.

Invasive paravenous transcutaneous techniques have the disadvantage of being invasive and cumbersome and requiring multiple points of entry. They have the further disadvantage of usually interrupting the vein only at specific points while omitting treatment of intermediate segments. Invasive endovenous techniques, although well suited for the treatment of large, straight, deeper veins, are difficult to use in the treatment of very small, convoluted superficial venous pathology, due to the difficulty of cannulation and the size of laser fibers.

Electrocoagulation techniques use monopolar or bipolar electrodes placed through the skin in contact with the vein, or inside the lumen of the vein, to coagulate and destroy it. Parvulesco, U.S. patent Publication No. 0,633,003A1, discloses the use of monopolar electrodes. The electrodes are introduced through a minimal cutaneous incision to coagulate and destroy a segment of vein. Ellman et al., U.S. Pat. No. 5,695,495, deliver electrodes from below the skin. Ellman et al describe a monopolar electrode placed inside the vein to coagulate and collapse a segment of vein.

Electrocoagulation techniques are invasive, non-vein specific and cause complications with perivenous tissue. Heating and destruction of such tissue results in scarring and paresthesias, in addition to electrical burns at the second electrode when monopolar techniques are used.

Chan, et al, U.S. Pat. No. 6,275,726 and Vargas, et. al., U.S. Patent Application Publication 2006/0069166 A1, disclose methods for improving laser light penetration through the skin and into tissues being treated. They describe injecting glycerol and other hypertonic or hyperosmotic solutions to the veins or tissues being treated to cause optical clearing of those tissues and make them more translucent, and, at the same time to slow the local venous flow to achieve better laser vein closure results.

Abels, et. al., Canadian Patent No. 2,326,071 disclose a method for introducing an exogenous solution into biological tissues that acts as a chromophore for absorbing specific wavelengths of light. Abels discusses the use of exogenous chromophores delivered to the patient prior to the light treatment. Abels also discloses an apparatus for measuring the concentration of the exogenous chromophore in the patient's body, and calibrates the light intensity to correspond to the concentration of the chromophore.

### OBJECT OF THE INVENTION

It is a primary object of this invention to provide an improved procedure to treat unwanted varicose and spider veins of various sizes and in various locations while reducing unwanted side effects with minimal trauma to the patient.

It is a further object of this invention to avoid the many disadvantages and problems encountered when using invasive laser techniques and electrocoagulation techniques.

It is a further object of the invention to reduce the side effects of percutaneous light treatments, including, in particular, hypo- and hyperpigmentation of the skin.

It is still a further object of the invention to provide a method for removing varicose, reticular or telangiectases veins, superficial venous branches or venectasia in patients in a way which achieves results in the shortest amount of time with minimal patient trauma.

It is a further and more specific object of this invention to build upon the success of sclerotherapy methods in treating these unwanted veins.

It is a still more specific object of this invention to combine light treatments with sclerotherapy to obtain synergisms which have heretofore not been achieved.

It is a related and specific object of this invention to provide kits to facilitate the practice of the methods of the invention.

### BRIEF SUMMARY OF THE INVENTION

The invention is a method for treatment of superficial venous pathology. The method comprises the steps of: (a) percutaneously piercing a vein to be treated with a needle and removing the needle from the patient thereby leaving an opening in the patient's skin and a needle track from the opening to the lumen of the vein and thereby causing a volume of tissue surrounding the needle track and the segment of vein with the needle holes and the segment of vein itself to undergo changes including becoming suffused with extravasated blood, the changes in the volume of tissue being manifested at the surface of the patient's skin by an affected area surrounding and including the opening in the skin which exhibits all or some of the physical, chemical and/or color changes of the affected volume of tissue; and (b) directing laser light at the patient's skin predominantly within the affected area of skin, the laser light being of such predetermined wavelength that at least a portion of it is absorbed by hemoglobin and/or oxyhemoglobin in extravasated blood suffused in the volume of tissue in which changes have occurred, to facilitate degradation of tissue in the affected volume and of said blood vessel.

In another embodiment of the invention, the step of percutaneously piercing the vein includes introducing a biocompatible, exogenous chromophore into the affected volume of tissue, i.e. a foreign substance having a specific, desired light-absorption characteristic, in order to facilitate the absorption of laser light and cause degradation of tissue in the affected volume and of the blood vessel.

A preferred method of the invention comprises the steps of: (a) percutaneously piercing a vein to be treated with a sclerotherapy needle; (b) introducing a sclerosing agent through the needle into the vein; (c) removing the sclerotherapy needle from the patient thereby leaving an opening in the patient's skin and a needle track from the opening in the patient's skin to the lumen of the vein and causing a volume of tissue surrounding the needle track and the segment of vein with the needle holes and the segment of vein itself to undergo changes, including becoming suffused with extravasated blood and sclerosing agent, the changes being manifested at the surface of the patient's skin by an affected area of skin surrounding and including the opening in the skin which exhibits all or some of the physical, chemical and/or color changes of the affected volume of tissue; and (d) directing laser light at the patient's skin substantially exclusively within the affected area of skin, the laser light being of such predetermined wavelength that at least a portion of it is absorbed by hemoglobin and/or oxyhemoglobin as well as water or other chromophores in extravasated blood and sclerotherapy solution suffused in the volume of tissue, to facilitate degradation of tissue in the affected volume and of said blood vessel.

In another embodiment of the invention, a biocompatible, exogenous chromophore can be introduced together with the sclerosing agent into the affected volume of tissue.

A further preferred method based upon the use of sclerosing agent includes such methods using a non-hypertonic, non-hyperosmotic sclerosing agent chosen from among agents which are detergents, chemical irritants, corrosives and toxins.

The methods of the invention which include sclerotherapy achieve an improvement in the success rate of vein removal relative to sclerotherapy treatment alone, percutaneous laser treatment alone, and, known methods which combine sclerotherapy and laser light treatments.

### IN THE DRAWINGS

FIG. 1 is a cross-sectional view through the skin of a patient.

FIG. 2 is the cross-sectional view of FIG. 1 showing a needle inserted into a segment of a reticular vein.

FIG. 3 is the cross-sectional view of FIG. 2 after withdrawal of the needle showing the volume of tissue affected by insertion and withdrawal of the needle.

FIG. 4 is a top view of the patient's skin in FIG. 3 following the insertion and withdrawal of the needle.

FIG. 5 is the cross-sectional view of FIG. 3 showing the laser treatment.

FIG. 6 is the cross-sectional view of FIG. 2 showing injection of sclerotherapy solution.

FIG. 7 is a top view of the patient's skin in FIG. 6 following the injection of sclerotherapy solution and withdrawal of the needle.

FIG. 8 is the cross-sectional view of FIG. 6 following the sclerotherapy treatment and withdrawal of the needle and showing the volume of tissue affected by the sclerotherapy treatment.

FIG. 9 is the cross-sectional view of FIG. 7 after laser treatment has been completed.

FIG. 10 is a cross-sectional view through the skin of a patient showing the insertion of a sclerotherapy needle into a segment of a telangiectasias.

FIG. 11 a the top view of the patient's skin in FIG. 10 following sclerotherapy and withdrawal of the needle.

FIG. 12 is the cross-sectional view of FIG. 10 after sclerotherapy has been completed.

FIG. 13 is the cross-sectional view of FIG. 12 after laser treatment has been completed.

FIG. 14 is a graph of the light absorption characteristics of hemoglobin (Hb), oxyhemoglobin (HbO₂) and melanin as a function of wavelength.

### DETAILED DESCRIPTION OF THE INVENTION

In its broadest embodiment, the invention is in a method for treating superficial venous pathology in a patient comprising the steps of: (a) percutaneously piercing a vein to be treated with a needle and removing the needle; and (b) directing laser or intense pulse light at the patient's skin predominantly within the area of skin manifesting physical, chemical and/or color changes caused by step (a). The wavelength of the light is matched to the light absorption characteristics of one or more endogenous or exogenous chromophores in the affected volume of tissue outside the lumen of the affected segment of vein.

In another embodiment the invention comprises the steps of: (a) percutaneously piercing a vein to be treated with a needle and removing the needle from the patient thereby leaving an opening in the patient's skin and a needle track from the opening to the lumen of the vein and thereby causing a volume of tissue surrounding the needle track and the segment of vein with the needle holes and the segment of vein itself to undergo changes including becoming suffused with extravasated blood, the changes in the affected volume of tissue being manifested at the surface of the patient's skin by an affected area surrounding and including the opening in the skin which exhibits all or some of the physical, chemical and/or color changes of the affected volume of tissue; and (b) directing laser light at the patient's skin substantially entirely within the affected area of skin, the laser light being of such predetermined wavelength that it is at least in part absorbed by hemoglobin and/or oxyhemoglobin in extravasated blood in the volume of tissue in which changes have occurred, to facilitate degradation of tissue in the affected volume and of said blood vessel.

In another embodiment, in order to facilitate the absorption of light by the patient's tissue, a biocompatible, exogenous chromophore may be introduced into the patient's tissue during the piercing steps.

More specifically, the method comprises the steps of: (a) percutaneously piercing a vein to be treated thereby creating an opening in the patient's skin and a track from the opening to the lumen of the vein and thereby causing an affected volume of tissue surrounding the needle track and the segment of vein with the needle holes and the segment of vein itself to undergo changes, the changes being manifested at the surface of the patient's skin by an affected area surrounding and including the opening in the skin which exhibits all or some of the physical, chemical and/or color changes of the affected volume of tissue; and (b) directing light of predetermined wavelength at the patient's skin predominantly within the affected area of skin, the predetermined wavelength being matched to the light absorption characteristics of one or more substances in the volume of tissue which has undergone changes, to facilitate degradation of said blood vessel.

When the vein is pierced with a needle and the needle is removed, those steps leave an opening in the patient's skin and a needle track and cause a volume of tissue surrounding the needle track to undergo changes including becoming suffused with extravasated blood. The changes in the volume of tissue are manifested at the patient's skin by an area of skin exhibiting ecchymosis.

The wavelength of the light which is used is predetermined so that at least a portion of the light directed at the patient's skin is absorbed by hemoglobin and/or oxyhemoglobin in extravasated blood suffused in the volume of tissue that has undergone changes.

In another embodiment, the invention is in a laser-assisted, sclerotherapy method for treating superficial venous pathology in a patient comprising the steps of: (a) percutaneously performing sclerotherapy on a vein to be treated; and (b) directing laser light of 400-1500 nm wavelength at the patient's skin predominantly within the area of skin manifesting some or all of the physical, chemical and/or color changes caused by step (a).

In a preferred laser-assisted sclerotherapy method, the invention comprises the steps of: (a) percutaneously piercing a vein to be treated with a sclerotherapy needle; (b) introducing a sclerosing agent through the needle into the vein; (c) removing the sclerotherapy needle from the patient thereby leaving an opening in the patient's skin and a needle track from the opening to the lumen of the vein and causing an affected volume of tissue surrounding the needle track and the segment of vein with the needle holes and the segment of vein itself to undergo changes, including its becoming suffused with extravasated blood and sclerosing agent, the changes in the volume of tissue being manifested at the surface of the patient's skin by an affected area of skin surrounding and including the opening in the skin which exhibits all or some of the physical, chemical and/or color changes of the affected volume of tissue; and (d) directing laser light at the patient's skin substantially entirely within the affected area of skin, the laser light being of such predetermined wavelength that it is at least in part absorbed by hemoglobin and/or oxyhemoglobin as well as water or other chromophores in extravasated blood and sclerotherapy solution suffused in the volume of tissue, to facilitate degradation of tissue in the affected volume and of said blood vessel.

A biocompatible, exogenous chromophore may be introduced together with the sclerosing agent.

Advantageous results are achieved in sclerotherapy methods in which the sclerosing agent is a non-hypertonic, non-hyperosmotic substance chosen from among sclerosing agents which are detergents, chemical irritants, corrosives and toxins.

### DEFINITIONS

The term percutaneous refers to procedures performed through the skin.

The term “affected area of skin” refers to the area of the skin surrounding and including the opening in the skin caused by a piercing instrument, e.g. a needle, which exhibits all or some of the physical, chemical or color changes in the underlying volume of tissue affected by the procedure.

The terms “ . . . physical, chemical or color changes . . . ” in the affected area of the skin refer to the changes which take place at and around a needle puncture site in the skin of a patient whether or not sclerotherapy has been performed and include the changes in the underlying affected volume of tissue some or all of which are manifested in the affected area of skin. These include ecchymosis, swelling and related color and chemical changes as well as any changes caused by introduction of sclerosing agent or exogenous chromophore.

The term “affected volume of tissue” includes tissue surrounding the needle track and the segment of vein with the needle holes and the segment of vein itself from and including the affected area of the skin overlying the vein being treated.

By “suffuse” is meant the spreading of fluids, e.g. extravasated blood and/or sclerotherapy solution, into the volume of tissue.

The term “ . . . light of predetermined wavelength . . . matched to the light absorption characteristics . . . ” means that a wavelength is selected to ensure that at least a portion of the light is absorbed to an effective degree by one or more substances, e.g. hemoglobin, oxyhemoglobin, water, sclerosing agent or exogenous chromophore, suffused in the volume of tissue. It is important to the objectives of the invention, namely the degradation of the segment of vein being treated, that the light be absorbed to an effective degree, i.e. that a significant amount of light energy be absorbed and converted to heat energy. Concomitantly, it is important that light not be absorbed preferentially in such skin tissue components as melanin and thereby cause burning and scarring.

By “chromophore” is meant a substance which has a specific light-absorption characteristic.

The term “predominantly” means that at least 70% and preferably 80% or more of the light directed at the patient's skin is directed at the affected area of the skin.

The term substantially entirely means that at least 90% and preferably 95% or more of the light is directed at the affected area of the skin.

### Laser-Assisted Sclerotherapy Methods

In a preferred embodiment, the invention is a laser-assisted sclerotherapy method for the treatment of superficial venous pathology such as superficial venous branches, reticular veins, venectasia, and telangiectases. Sclerotherapy is performed on the unwanted vein by known procedures using known sclerotherapy needles and one or more known sclerotherapy agents. Suitable sclerotherapy needles are 27-30 G and may be obtained from the Becton-Dickenson Company. Other suitable needles may be used.

A variety of known sclerotherapy agents can be used depending on the specific pathology sought to be treated. Commonly used agents include Sotradecol, Polydecanol, or a solution of glycerin and Lidocaine.

Weiss, et al., Vein Diagnosis and Treatment, McGraw Hill 2001, describe a spectrum of sclerotherapy agents. These agents are commonly classified into three broad categories, including (a) hypertonic or hyperosmotic agents, (b) detergent sclerosing agents, and (c) chemical irritants, alternatively referred to as corrosives or toxins. The disclosure of Weiss et al. is hereby incorporated by reference. Advantageous results are achieved in particular with the sclerosing agents in categories (b) and (c).

The steps of insertion of the sclerotherapy needle through the patient's skin and into the unwanted vein, introduction of the sclerosing agent, and withdrawal of the needle all cause extravasation blood and sclerosing agent to suffuse into an affected volume of tissue. Other physical and biological changes, such as localized swelling, occur in that volume of tissue as a result of the injury caused by the insertion of the needle and injection of the sclerosing solution. The presence of the extravasated blood can be noted by the black and blue marks, ecchymosis, in the affected area of skin and in the minimal bleeding through the needle hole. Other changes, such as local swelling after sclerotherapy, differences in the concentration of water, electrolytes and other organic or inorganic substances or tissue breakdown in the volume of tissue are also present.

Specifically, the method includes percutaneously introducing a sclerotherapy needle through the skin of a patient into the lumen of the vein to be treated thereby creating an opening in the skin and a needle track extending from the opening to the lumen of the vein. A sclerosing agent from an appropriate syringe is then introduced into the needle and passes through the needle into the vein. The sclerotherapy needle is then removed from the patient. Laser or intense pulse light energy is then directed at the patient's skin predominantly at the affected area caused by the sclerotherapy treatment. In the preferred embodiment light energy is first applied to the distal edge of the affected area, and then is moved proximally towards the opening in the skin. This approach diminishes bleeding from the opening in the skin.

In preferred embodiments, immediately after sclerotherapy, or, after a suitable dwell time, laser or intense pulse light is directed at the patient's skin substantially entirely within the affected area of skin. The wavelength of the light is matched to the light absorption characteristics of one or more endogenous or exogenous chromophores in the affected volume of tissue, outside the lumen of the affected segment of vein.

The result of the procedure is an improvement in the success rate of vein removal relative to sclerotherapy or percutaneous laser treatment alone and relative to known methods which combine sclerotherapy and laser light treatments. The method takes advantage of the physical, chemical and color changes in the volume of tissue which are manifested in the affected area of skin. Because the laser energy is directed only at the affected area of skin, and not along the entire course of the vein, total light energy used is reduced and lasting cutaneous complications of percutaneous laser treatment of veins are not encountered. Likewise, given the light-absorption characteristics of the chromophores in the volume of tissue outside the lumen of the segment of vein being treated, only small amounts of laser energy are needed to produce the desired effect as compared to prior art percutaneous laser treatments of superficial venous pathology.

In the preferred embodiment advantage is taken of the presence of hemoglobin and oxyhemoglobin in the extravasated blood cells suffused in the volume of tissue. The presence of the hemoglobin can be visualized by observing an area of ecchymosis in the affected area of skin. The light energy is targeted to the ecchymotic area.

In still further detail, the method is one for treating, with minimum trauma, superficial venous pathology, comprising the steps of: (a) percutaneously introducing one or more sclerotherapy needles through the skin of the patient into the lumen of a vein to be treated at one or more locations along the vein, thereby creating one or more openings in the skin and one or more needle tracks extending from the opening(s) to the lumen of the vein; (b) introducing a sclerosing composition through the needle(s) into the vein at the location(s) along the vein; (c) removing the sclerotherapy needle(s) from the patient, steps (a)-(c) causing extravasation of blood and sclerosing agent to suffuse in an affected volume of tissue as well as other physical, chemical and structural changes, such as local swelling; (d) removing superficial extraneous blood from the skin around the openings in the skin at the multiple locations; (e) optionally permitting the patient to dwell prior to laser treatment for not longer than the time in which the color changes remain before dissipation or for not longer than the time in which the other physical, structural and chemical changes exist; and then (f) directing laser light at the patient's skin predominantly in the affected area of skin.

In another embodiment, steps (a), (c), (d), (e) and (f) can be performed. The extravasation of blood will occur and can be targeted by the light energy regardless of whether or not sclerotherapy solution is introduced. The light is directed at the affected area of the skin.

An exogenous chromophore may be introduced into the patient's tissue either in the percutaneous piercing step or in a subsequent step prior to directing light at the patient's skin.

Advantageously, a composition comprising a sclerosing agent and an effective amount of an exogenous chromophore can be used. In that circumstance, the light energy treatment is also directed predominantly at the affected area of skin. The wavelength of the light is chosen in part to match the light-absorption characteristics of the exogenous chromophore.

### DESCRIPTION OF THE DRAWINGS

With reference to the drawings, in FIG. 1 reference numeral 10 refers to a cross-section of a patient's skin and subcutaneous layers comprising epidermis 12, papillary dermis 14, reticular dermis 16, subcutaneous layer 18, reticular vein 20 and telangiectasias 22.

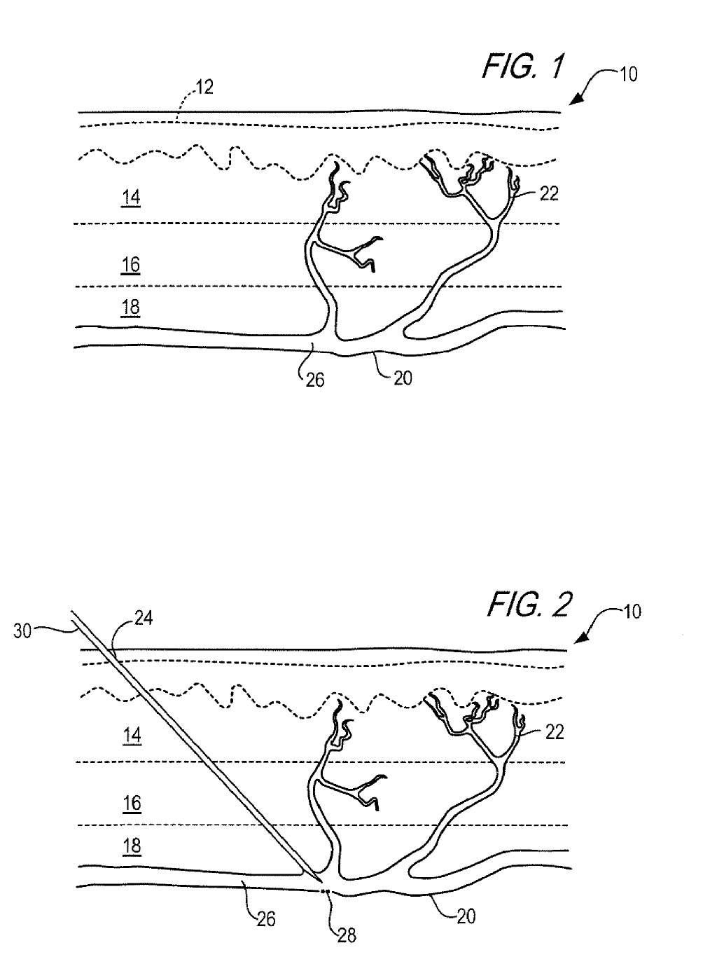

In FIG. 2 reference numeral 30 depicts a needle, which may or may not be hollow as shown, inserted through the skin creating opening 24. Needle 30 passes through the subcutaneous layers into lumen 26 of reticular vein 20. Occasionally, the needle, if advanced too far, may create a second puncture in vein 20 as shown by reference numeral 28.

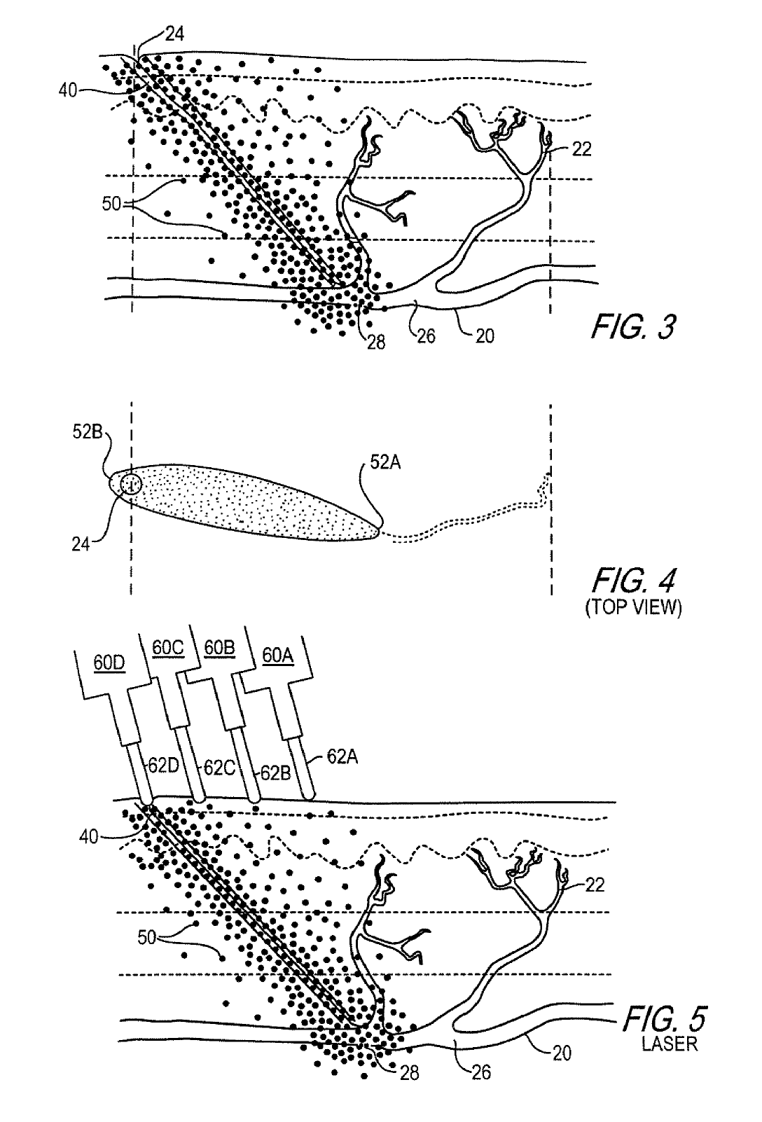

FIG. 3 shows the affected volume of tissue as defined above, which has been affected by inserting and removing the needle. Extravasation of red blood cells 50 takes place within that volume as a consequence of the insertion and withdrawal of the needle from needle track 40 and vein 20.

FIG. 4 shows the area of skin affected by the procedure, including skin opening 24 and ecchymotic area 52 having a proximal edge 52B and a distal edge 52A. The changes in the affected volume of tissue are manifested in the affected area of FIG. 4.

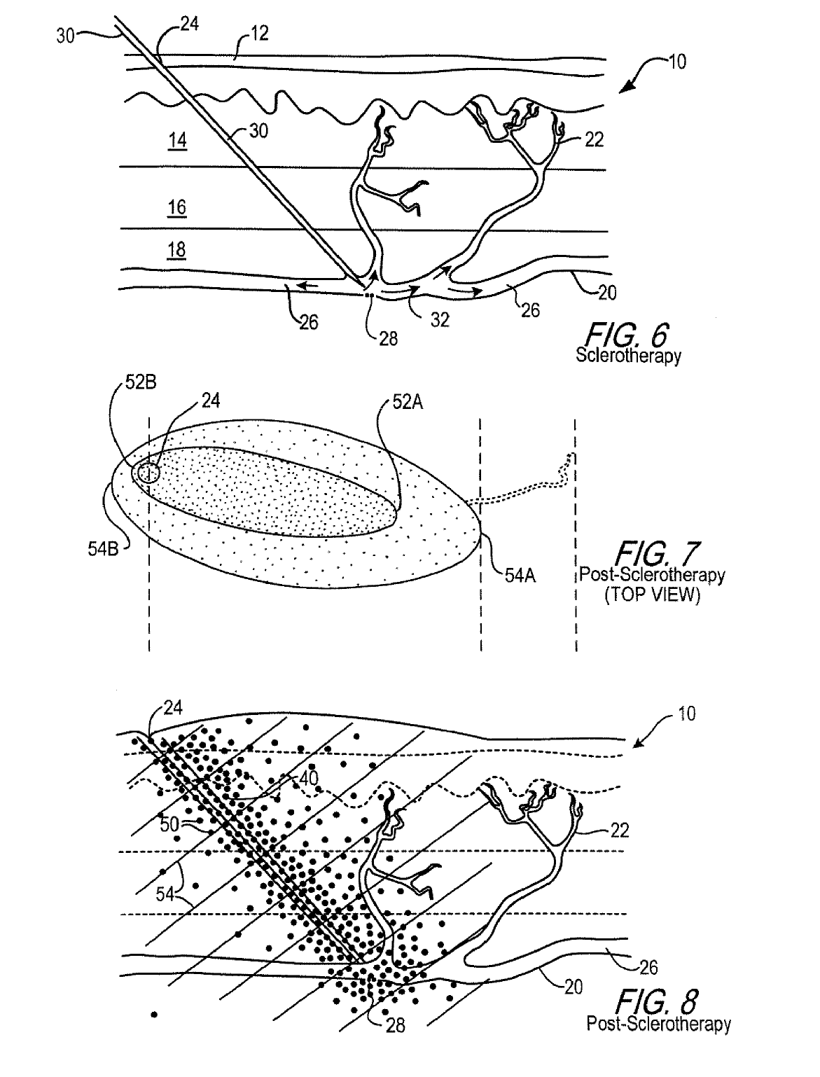

In FIG. 5, a laser source 60 emits a beam of light 62. Light source 60 is positioned first at position 60A, the distal end of the affected area of skin showing ecchymotic changes. Light beam 62 travels through the skin and is absorbed in the affected volume of tissue by the hemoglobin and oxyhemoglobin present in extravasated red blood cells 50. Depending on the diameter of the tip of the light source, it can be moved about, preferably from the distal edge towards the skin opening 24, i.e., from point 62A to 62D, to ensure that all affected areas of skin changes are subjected to the light treatment at several points.

FIG. 6, shows the introduction of sclerotherapy solution 32 into the vein through needle 30, after it is inserted as depicted in FIG. 2.

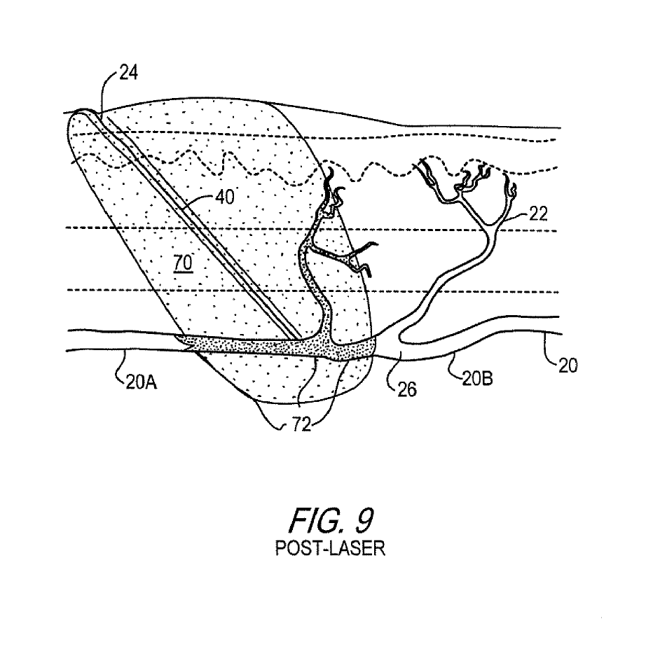

FIG. 7 shows the area of skin affected by the sclerotherapy procedure, including opening in the skin 24, ecchymotic area 52 having a proximal edge 52B and distal edge 52A and a localized area of swelling 54 having a proximal edge 54B and a distal edge 54A. The entire area of FIG. 7 manifests the changes which have occurred in the affected volume of tissue.

FIG. 8 shows the volume of tissue affected by the sclerotherapy procedure, including extravasated red blood cells 50 and localized swelling 54 and suffused sclerotherapy solution in and immediately around needle track 40 and vein 20. Vein 20 has been bathed by the sclerotherapy solution which may result in swelling of the vein.

After the sclerotherapy treatment, the patient is subjected to light energy treatment as depicted in FIG. 5. FIG. 9 shows the area of changes in the affected volume of tissue that result from the light treatment. The tissues at and around needle track 40 are degraded as shown at 70. Reticular vein 20 is partially or totally interrupted and degraded, at 72, affecting the flow of blood from segment 20A to segment 20B. The tissue degradation following light treatment occurs whether the patient was subjected to sclerotherapy treatment as shown in FIG. 6, or subjected only to needle insertion and removal as depicted in FIGS. 1-2.

In FIG. 10, sclerotherapy needle 30 has been inserted through the skin layer into the lumen 82 of vein 80 at puncture site 84, thereby creating skin opening 24. A sclerotherapy solution 32 is introduced through the hollow sclerotherapy needle.

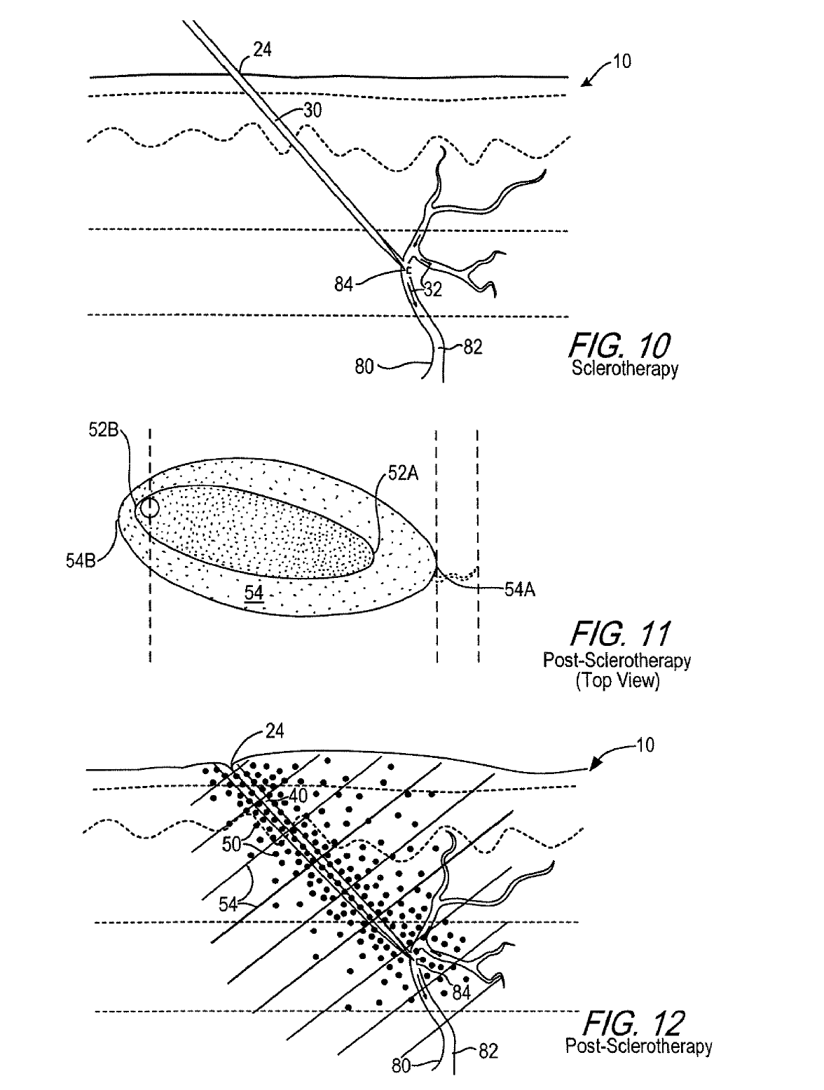

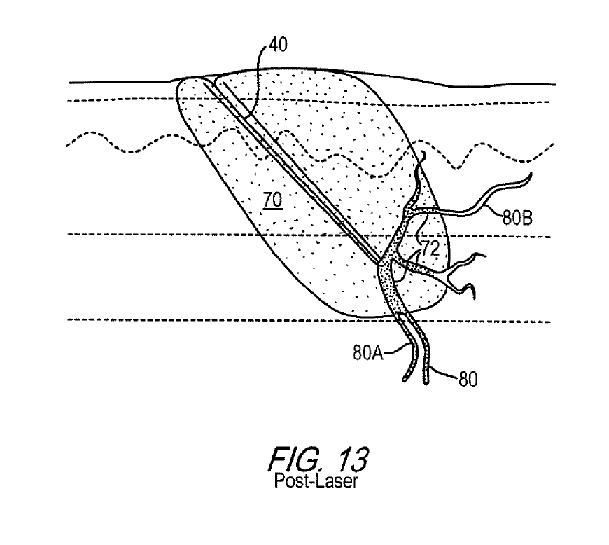

FIGS. 11 and 12 show the extravasation of red blood cells and sclerotherapy solution in the affected volume of tissue following the insertion of the needle, introduction of the sclerotherapy solution and withdrawal of the needle as well as local tissue and skin swelling.

After the sclerotherapy treatment, the patient is subjected to light energy treatment as depicted in FIG. 5. FIG. 13, shows the area of changes in the affected volume of tissue that result from the light treatment. The tissues at and around needle track 40 are degraded as shown at 70. Vein 80 is partially or totally interrupted and degraded, at 72, affecting the flow of blood from segment 80A to segment 80B.

Proper and practical implementation of the method of the invention requires a complete evaluation and diagnosis of the patients' venous disorder and design of a treatment plan.

Involvement and insufficiency of major venous trunks as well as all other sources of reflux should first be treated by any of the appropriate current techniques, surgical or endovenous, prior to the treatment of the dependent superficial venous pathology. Once treatment of the main trunks and sources of reflux have been successfully achieved, sclerotherapy for the superficial venous pathology may be commenced. When this superficial venous pathology is not associated with significant main trunk involvement and other obvious sources of reflux, the sclerotherapy treatment may start immediately.

Sclerotherapy treatments are well known to those skilled in the art. A variety of sclerosing agents in different strengths and volumes may be used as described in Weiss, et al. The combination of different agents and their strengths will depend on vein size, depth, pressure, skin color as well as practitioner preference and experience. Sclerotherapy should always start with the larger deeper veins, the reticular and feeder veins, as well as small perforators, the object being to decrease the venous flow and venous hydrostatic pressure of the smaller and more superficial venous manifestations. The decrease in hydrostatic venous pressure is a prerequisite for successful therapy, as well as for reducing unwanted side effects.

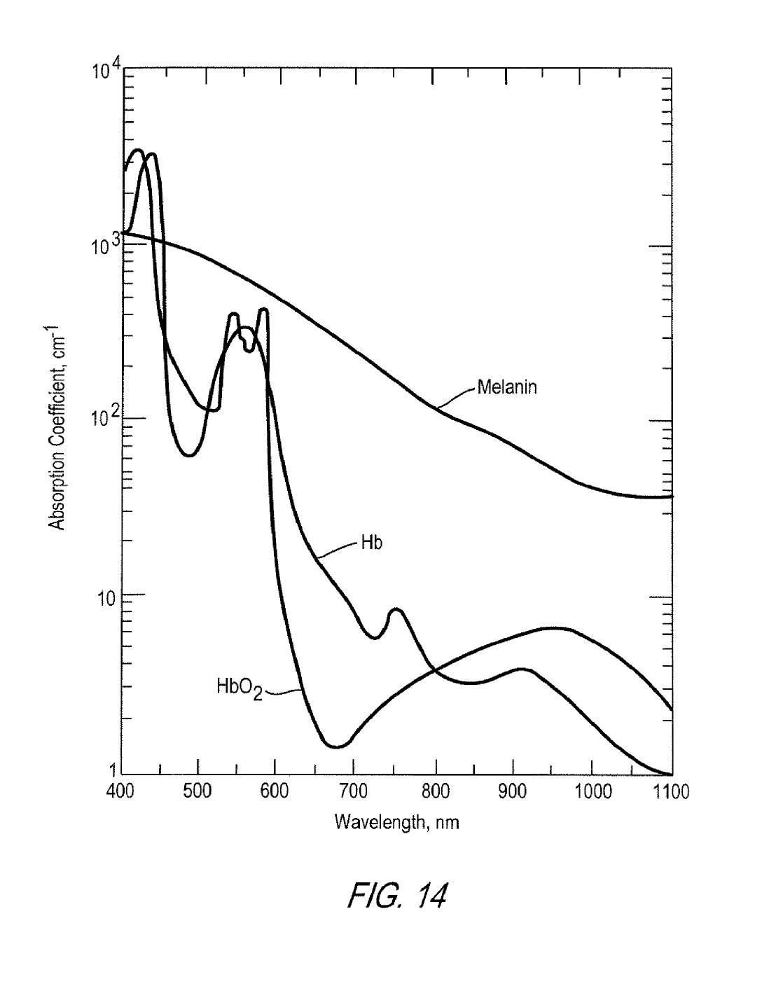

FIG. 14 shows the light-absorption characteristics of Hb, HbO₂ and H₂O as a function of wavelength. This permits the physician or technician to match the wavelength of the laser or light source to the light absorption when using these components as chromophores.

When all main trunk pathology has been treated by the appropriate technique, and when all secondary, large, reticular and perforator veins feeding a particular area of superficial venous pathology have been treated by sclerotherapy or other methods, e.g., mini-phlebotomy, the superficial venous pathology may be treated by the methods of the invention.

Sclerotherapy of the superficial venous pathology area is performed in the usual manner by multiple injections of the appropriate sclerosing solution(s) in the concentrations and amounts dictated by the circumstances, every few millimeters along the superficial reticular and feeder veins, and telangiectases.

The light energy treatment, which includes laser as well as intense pulse light, provides light energy at one or a multiplicity of wavelengths. The wavelength(s) and intensities are matched to the light-absorption characteristics of the chromophores in the affected volume of tissue, in particular hemoglobin and oxyhemoglobin. It is important that at least a portion of the light be absorbed by these blood chromophores.

Hemoglobin and oxyhemoglobin in the red blood cells are chromophores for a range of laser wavelengths from less 300 nm to more than 1100 nm water and other exogenous substances are likewise chromophores for a range of laser wavelengths. In one embodiment advantage is taken of the 810-980 nm wavelength. This wavelength is preferentially chosen because at this wavelength the absorption by hemoglobin is sufficient to achieve the desired result, but is not so high as to cause almost complete absorption at the very superficial layer thus preventing the penetration and efficacious treatment of the deeper layers.

Satisfactory results are achieved with light having a wavelength broadly within the range of 400-1500 nm. Preferably the light is at a wavelength between 750-1320 nm.

In one embodiment of the invention light treatment immediately follows the sclerotherapy treatment. This takes maximum advantage of the hemoglobin in the red blood cells in and around the venous segment, the needle track and the opening in the skin. However, some dwell time may be optionally given provided that the dwell time does not exceed the time that it takes for the relevant physical, chemical and color changes to which the light energy is targeted to dissipate.

Light energy is directed at the affected area of the skin. The affected area in which relevant physical, chemical and color changes are present is generally only a few square millimeters. It surrounds and includes the opening in the skin. The size of the area may vary depending on the type and location of pathology and characteristics of the sclerotherapy treatment performed. Directing light energy at the affected area results in carbonization and degradation of any blood in the needle track and surrounding tissues, which may result in observing a slightly grayish tinge through the skin. The procedure is relatively painless and may result in a pinprick sized scab which lasts for 1 to 3 weeks which heals without scarring.

It is important, prior to the light energy treatment step, to wipe any excess blood from the opening in the skin of the needle track to maximize penetration of light and minimize absorption of light by the excess blood with attendant superficial carbonization and increased scabbing.

Light source operation depends on the patient's condition, the light used and other considerations. In one embodiment, the light is discharged in a sequence of multiple discharges broadly from 3-9 and typically about 5, at the opening in the skin of the needle track and immediate surrounding skin area, where the relevant color changes are present, as well as the other local physical, structural and chemical changes, at from 5 to 10 watts depending on local skin conditions and usually at 8 watts for 0.1 to 0.2 seconds at 0.1 to 0.2 second intervals. A 0.7 to 2 mm handpiece may be used. The size will depend on the particular pathology, size, and location of the vein being treated. Immediately after the light discharge(s) one can observe a tiny amount of carbonization at the opening of the needle track if any blood is present at the opening, with a small temporary grayish tinge in the skin surrounding the opening.

After the light treatment of all the openings in the skin and their surrounding areas as described above, the treated area is dressed in the usual manner as after sclerotherapy. A small amount of 0.1% hydrocortisone cream and cotton balls are applied to the area, which is then wrapped with an ace bandage for 2-6 hours. A compression stocking, less than Class-1 or Class-1, is usually recommended to be worn overnight.

Patients return in 1-2 weeks or other appropriately varied intervals at which point any small scabs covering the openings of the needle tracks have or are in the process of falling off. At this point additional sclerotherapy may be performed as necessary to remove any remnants of the superficial venous pathology. The light treatment may also be performed again if necessary, though in most circumstances repeat treatment is not expected.

### EXAMPLE

### Actual Treatment of Patient

When all main trunk pathology has been treated by the appropriate technique, as well as all dependant, large reticular and perforator veins feeding a particular area of superficial venous pathology have been treated by sclerotherapy or other methods (mini-phlebectomy), the methods of the invention may be performed.

Sclerotherapy of the area of superficial venous pathology is performed in the usual manner: To treat reticular and feeder veins Sotradecol solution 0.3% is drawn in a 3 cc Becton Dickinson (BD) syringe. Using a 27-G-½ BD needle, sequential injections of 0.2-0.5 cc of the solution are injected along the length of the veins every 0.5-2 cm. To perform sclerotherapy of the telangiectatic veins, 0.1% Sotradecal, 0.3% Polydecanol, or 70% glycerin and lidocaine solution is drawn into a 3 cc BD syringe. Using a 30-G-½ BD needle sequential injections of 0.1-0.4 cc of solution are made every 0.2-0.5 cm along the length of the telangiectases.

A few minutes after sclerotherapy has been performed areas of local swelling of the skin a few millimeters in radius around the openings in the skin are visible. Also visible within these areas of localized swelling are elongated, elliptical areas of ecchymosis of the skin encompassing the needle track opening and extending for a few millimeters in the direction in which the needle has been introduced. This ecchymotic area represents extravasation of red blood cells in tissues around the sclerotherapy needle track and in tissues around the local segments of the vein with the needle hole(s).

Immediately after the laser discharge(s) one can see a spec of carbonization at the skin opening of the needle track. It is important, prior to the laser discharge, to wipe any excess blood from the skin opening of the needle track, to maximize penetration of the laser and minimize absorption of the laser energy by this excess blood which results in unnecessary, very superficial carbonization and increased scabbing.

At this point, laser treatment is performed using an 810 nm diode laser from Diomed Corp. with a 2 mm hand piece or a 980 nm diode laser from Vascular Solutions Corp. with a 0.7 mm hand piece.

The lasers use between 5-10 watts depending on local skin conditions and typically use 8 watts with 0.1-0.2 second pulse duration and 0.1-0.2 second pulse interval.

Approximately 5 discharges at these settings are pointed, at a slight angle from perpendicular to the skin, into the needle hole and surrounding skin area showing the underlying tissue changes. The sequential laser discharges are started at the most distal end of the ecchymotic patch progressing up to the needle hole.

The kits of the invention may include two or more of the basic tools for carrying out the methods of the invention. The basic tools are (a) sclerotherapy needles; (b) syringes; (c) sclerotherapy solution; (d) a disposable light source handle; (e) a laser fiber; and (f) materials for dressing the wound area post-procedure.

In one embodiment of a basic two-component kit, the kit includes a disposable handle for a laser fiber and a sclerotherapy solution.

In another embodiment of a basic two-component kit, the kit includes one or more disposable laser fibers and a sclerotherapy solution.

In one embodiment of a basic three-component kit, the kit includes a disposable handle for a laser fiber, one or more disposable laser fibers and a sclerotherapy solution.

These basic, two-component and three-component kits may further include one or more of the other components, e.g. (a) a plurality of sclerotherapy needles, (b) a plurality of syringes and/or (c) materials for dressing the wounds, post-procedure.

By way of illustration, not limitation, representative kits may contain:
(1) a disposable handle, a sclerotherapy solution, and a plurality of sclerotherapy needles.
(2) one or more laser fibers, a sclerotherapy solution, and a plurality of sclerotherapy needles.
(3) a disposable handle, a sclerotherapy solution and a plurality of syringes.
(4) one or more laser fibers, a sclerotherapy solution, and a plurality of syringes.

In an embodiment containing four components, the kit contains a plurality of sclerotherapy needles, a plurality of syringes, a scierotherapy solution and a disposable laser handle. Other post-procedure components such as cotton swabs and the like and bandages can also be included in the kits.

I claim:

1. A method for treating superficial venous pathology in a patient comprising the steps of:
(a) percutaneously piercing a vein to be treated thereby creating an opening in the patient's skin and a track from said opening to the lumen of said vein and thereby causing changes in an affected volume of tissue surrounding the needle track and the segment of vein with the needle holes and the segment of vein itself, said changes being manifested at the surface of the patient's skin by an affected area surrounding and including said opening in the skin which exhibits all or some of the physical, chemical and/or color changes of the affected volume of tissue; and
(b) directing light of predetermined wavelength at said patient's skin predominantly within said affected area of skin, said predetermined wavelength being matched to the light absorption characteristics of one or more substances in said volume of tissue to facilitate degradation of said blood vessel.

2. A method as recited in claim 1 wherein said vein is pierced with a needle and said needle is removed leaving an opening in the patient's skin and a needle track and causing changes in said affected volume of tissue, including its becoming suffused with extravasated blood, said changes being manifested at the patient's skin by an area of skin exhibiting ecchymosis.

3. A method as recited in claim 1 wherein the wavelength of light is such that a portion of the light is absorbed by hemoglobin and/or oxyhemoglobin in extravasated blood suffused in the affected volume of tissue.

4. A method as recited in claim 1 wherein a biocompatible, exogenous chromophore is introduced into said patient's tissue before light is directed of said patient's skin.

5. A method for treating superficial venous pathology in a patient comprising the steps of:
(a) percutaneously piercing a vein to be treated with a needle and removing said needle from the patient thereby leaving an opening in the patient's skin and a needle track from said opening to the lumen of said vein and thereby causing an affected volume of tissue surrounding said needle track and the segment of vein with the needle holes and the segment of vein itself to undergo changes including its becoming its suffused with extravasated blood, said changes in the volume of tissue being manifested at the surface of the patient's skin by an affected area surrounding and including said opening in the skin which exhibits all or some of the physical, chemical and/or color changes of the affected volume of tissue;
and
(b) directing laser light at said patient's skin substantially entirely within said affected area of skin, said laser light being of such predetermined wavelength that at least a portion of it is absorbed by hemoglobin and/or oxyhemoglobin in extravasated blood suffused in said volume of tissue in which changes have occurred, to facilitate degradation of said blood vessel.

6. A method for treating superficial venous pathology in a patient comprising the steps of:
(a) percutaneously piercing a vein to be treated with a sclerotherapy needle;
(b) introducing a sclerosing agent through said needle into said vein;
(c) removing said sclerotherapy needle from said patient thereby leaving an opening in the patient's skin and a needle track from said opening to the lumen of said vein and causing changes in an affected volume of tissue surrounding the said needle track and the segment of vein with the needle holes and the segment of vein itself including its becoming suffused with extravasated blood and sclerosing agent, said changes being manifested at the surface of the patient's skin by an affected area of skin surrounding and including said opening in the skin which exhibits all or some of the physical, chemical and/or color changes of the affected volume of tissue; and
(d) directing laser light at said patient's skin predominantly within said affected area of skin, said laser light being of such predetermined wavelength that at least a portion of it is absorbed by hemoglobin, and/or oxyhemoglobin and/or water in extravasated blood suffused in said volume of tissue, to facilitate degradation of said blood vessel.

7. A method as recited in claim 5 wherein said laser light is directed substantially entirely within said affected area of skin.

8. A method as recited in claim 5 wherein said laser light has a wavelength of between 400-1500 nm.

9. A method of recited in claim 6 wherein a biocompatible, exogenous chromophore is introduced into said patient's tissue together with said sclerosing agent.

10. A method as recited in claim 6 in which the sclerosing agent is a non-hypertonic, non-hyperosmotic agent chosen from among sclerosing agents which are detergents, chemical irritants, corrosives and toxins.

11. A method for treating superficial venous pathology in a patient comprising the steps of:
(a) percutaneously introducing a sclerotherapy needle though the skin of said patient into the lumen of a vein to be treated;
(b) introducing a sclerosing agent through said needle into said vein;
(c) removing said sclerotherapy needle from said patient and thereby leaving an opening in the patient's skin and a needle track extending from said opening to the lumen of said vein and causing an affected area of skin surrounding and including said needle track to exhibit physical, chemical and/or color changes; and
(d) directing light of predetermined wavelength at the patient's skin predominantly within the affected area of the skin, the predetermined wavelength being matched to the light absorption characteristics of one or more substances in the affected volume of tissue.

12. A method of claim 6, wherein multiple skin openings and needle tracks are created by multiple introduction of sclerotherapy needles, each said opening and needle track being subjected to sclerotherapy and subsequently to light energy treatment by directing light at the patient's skin substantially entirely within said affected area of skin.

13. A method for treating superficial venous pathology in a patient comprising the steps of:
(a) percutaneously piercing a vein to be treated with a needle and removing said needle; and
(b) directing laser light or intense pulse light at said patient's skin predominantly within the area of skin manifesting physical, chemical and/or color changes caused by step (a).

14. A method as recited in claim 13 wherein the wavelength of said light is matched to the light absorption characteristics of one or more endogenous or exogenous chromophores in the affected volume of tissue, outside the lumen of the affected segment of vein.

15. A laser-assisted, sclerotherapy method for treating superficial venous pathology in a patient comprising the steps of:
(a) percutaneously performing sclerotherapy on a vein to be treated; and
(b) directing laser or intense pulse light at said patient's skin predominantly within the area of skin manifesting physical, chemical and/or color changes caused by step (a).

16. A method as recited in claim 15 wherein the wavelength of said light is matched to the light absorption characteristics of one or more endogenous or exogenous chromophores in the affected volume of tissue, outside the lumen of the affected segment of vein.

17. A kit for use in a percutaneous, laser-assisted, sclerotherapy method for collapsing varicose, reticular or telangiectatic veins, superficial venous branches or venectasia in a patient comprising: two or more components selected from (a) sclerotherapy needles; (b) syringes; (c) sclerotherapy solution; (d) a disposable light source handle; (e) a laser fiber; and (f) materials for dressing the wound area post-procedure.

18. A kit as recited in claim 17 comprising a disposable light source handle and a sclerotherapy solution.

19. A kit as recited in claim 18 wherein said light source handle is a laser handle.

20. A kit as recited in claim 17 comprising one or more disposable laser fibers and a sclerotherapy solution.

21. A kit as recited in claim 17 comprising a disposable handle for a laser fiber, one or more disposable laser fibers and a sclerotherapy solution.

22. A kit as recited in claim 17 comprising a disposable handle, a sclerotherapy solution, and a plurality of sclerotherapy needles.

23. A kit as recited in claim 17 comprising one or more laser fibers, a sclerotherapy solution, and a plurality of sclerotherapy needles.

24. A kit as recited in claim 17 comprising a disposable handle, a sclerotherapy solution and a plurality of syringes.

25. A kit as recited in claim 17 comprising one ore more laser fibers, a sclerotherapy solution, and a plurality of syringes.

26. A composition of matter useful for treating superficial venous pathology in a patient comprising:
(a) sclerotherapy solution; and
(b) an effective amount of biocompatible, exogenous chromophore.

27. A composition of matter as recited in claim 27 wherein said sclerotherapy agent is a non-hypertonic, non-hyperosmotic agent chosen from among sclerosing agents which are detergents, chemical irritants, corrosives and toxins.
